SEQUENCE LISTING (14 sequences)

SEQ ID NO: 1 - DNA, 1623 bp, Escherichia coli
  CDS (1)..(1620): pckA
atgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcagctgattggcggcacctggtacggcggcgaaatgaagaaagggatgttctcgatgatgaactacctgctgccgctgaaaggtatcgcttctatgcactgctccgccaacgttggtgagaaaggcgatgttgcggtgttcttcggcctttccggcaccggtaaaaccaccctttccaccgacccgaaacgtcgcctgattggcgatgacgaacacggctgggacgatgacggcgtgtttaacttcgaaggcggctgctacgcaaaaactatcaagctgtcgaaagaagcggaacctgaaatctacaacgctatccgtcgtgatgcgttgctggaaaacgtcaccgtgcgtgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaa

SEQ ID NO: 2 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 3 - DNA, 20 bp, artificial sequence
  synthetic DNA
gatccgagcctgacaggtta

SEQ ID NO: 4 - DNA, 20 bp, artificial sequence
  synthetic DNA
gcatgcgctcggtcaggtta

SEQ ID NO: 5 - DNA, 22 bp, artificial sequence
  synthetic DNA
aggcctgaagatggcactatcg

SEQ ID NO: 6 - DNA, 20 bp, artificial sequence
  synthetic DNA
ccggagaagcgtaggtgtta

SEQ ID NO: 7 - DNA, 1156 bp, Escherichia coli
  misc (1)..(1156): Mutagene DNA
ctagtaacggccgccagtgtgctggaattcggcttgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcaagccgaattctgcagatcctgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaagccgaattctgcagatatccatcacactggcggccgctcgagcatgcat

SEQ ID NO: 8 - DNA, 1294 bp, Escherichia coli
  misc (1)..(3): Start codon of delta pckA allele
atgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcaagccgaattctgcagatcctgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaa

SEQ ID NO: 9 - DNA, 21 bp, artificial sequence
  synthetic DNA
caggacgaggaactaactatg

SEQ ID NO: 10 - DNA, 20 bp, artificial sequence
  synthetic DNA
tcacgatggcggaatacaac

SEQ ID NO: 11 - DNA, 2111 bp, Escherichia coli
  RBS (8)..(12)
caggacgaggaactaactatgaatggcgcacagtgggtggtacatgcgttgcgggcacagggtgtgaacaccgttttcggttatccgggtggcgcaattatgccggtttacgatgcattgtatgacggcggcgtggagcacttgctgtgccgacatgagcagggtgcggcaatggcggctatcggttatgcccgtgctaccggcaaaactggcgtatgtatcgccacgtctggtccgggcgcaaccaacctgataaccgggcttgcggacgcactgttagattctatccctgttgttgccatcaccggtcaagtgtccgcaccgtttatcggcacggacgcatttcaggaagtggatgtcctgggattgtcgttagcctgtaccaagcacagctttctggtgcagtcgctggaagagttgccgcgcattatggctgaagcattcgacgttgccagctcaggtcgtcctggtccggttctggtcgatatcccaaaagatatccagctagccagcggtgacctggaaccgtggttcaccaccgttgaaaacgaagtgactttcccacatgccgaagttgagcaagcgcgccagatgctggcaaaagcgcaaaaaccgatgctgtacgttggtggtggcgtgggtatggcgcaggcagttcctgctttacgagaatttctcgctaccacaaaaatgcctgccacctgcacgctgaaagggctgggcgcagttgaagcagattatccgtactatctgggcatgctgggaatgcatggcaccaaagcggcgaacttcgcggtgcaggagtgcgacttgctgatcgccgtgggtgcacgttttgatgaccgggtgaccggcaaactgaacaccttcgcaccacacgccagtgttatccatatggatatcgacccggcagaaatgaacaagctgcgtcaggcacatgtggcattacaaggtgatttaaatgctctgttaccagcattacagcagccgttaaatatcaatgactggcagctacactgcgcgcagctgcgtgatgaacatgcctggcgttacgaccatcccggtgacgctatctacgcgccattgttgttaaaacaactgtcggatcgtaaacctgcggattgcgtcgtgaccacagatgtggggcagcaccagatgtgggccgcgcagcacatcgcacacactcgcccggaaaatttcattacctccagcggcttaggcaccatgggtttcggtttaccagcggcggttggcgcacaagtcgcacgaccgaacgatactgtcgtctgtatctccggtgacggctctttcatgatgaatgtgcaagagctgggcaccgtaaaacgcaagcagttaccgttgaaaatcgtcttactcgataaccaacggttagggatggttcgacaatggcagcaactgttttttcaggaacgatacagcgaaaccacccttactgataaccccgatttcctcatgttagccagcgccttcggcatccctggccaacacatcacccgtaaagaccaggttgaagcggcactcgacaccatgctgaacagtgatgggccatacctgcttcatgtctcaatcgacgaacttgagaacgtctggccgctggtgccgcctggcgccagtaattcagaaatgttggagaaattatcatgatgcaacatcaggtcaatgtatcggctcgcttcaatccggaaaccttagaacgtgttttacgcgtggtgcgtcatcgtggtttccacgtctgctcaatgaatatggctgccgccagcgatgcacaaaatataaatatcgaattgaccgttgccagcccacggtcggtcgacttactgtttagtcagttaaataaactggtggacgtcgcacacgttgccatctgccagagcacaaccacatcacaacaaatccgcgcctgagcgcaaaaggaatataaaaatgaccacgaagaaagctgattacatttggttcaatggggagatggttcgctgggaagacgcgaaggtgcatgtgatgtcgcacgcgctgcactatggcacctcggtttttgaaggcatccgttgctacgactcacacaaaggaccggttgtattccgccatcgtga

SEQ ID NO: 12 - protein, 548 aa, Escherichia coli
MNGAQWVVHALRAQGVNTVFGYPGGAIMPVYDALYDGGVEHLLCRHEQGAAMAAIGYARATGKTGVCIATSGPGATNLITGLADALLDSIPVVAITGQVSAPFIGTDAFQEVDVLGLSLACTKHSFLVQSLEELPRIMAEAFDVASSGRPGPVLVDIPKDIQLASGDLEPWFTTVENEVTFPHAEVEQARQMLAKAQKPMLYVGGGVGMAQAVPALREFLATTKMPATCTLKGLGAVEADYPYYLGMLGMHGTKAANFAVQECDLLIAVGARFDDRVTGKLNTFAPHASVIHMDIDPAEMNKLRQAHVALQGDLNALLPALQQPLNINDWQLHCAQLRDEHAWRYDHPGDAIYAPLLLKQLSDRKPADCVVTTDVGQHQMWAAQHIAHTRPENFITSSGLGTMGFGLPAAVGAQVARPNDTVVCISGDGSFMMNVQELGTVKRKQLPLKIVLLDNQRLGMVRQWQQLFFQERYSETTLTDNPDFLMLASAFGIPGQHITRKDQVEAALDTMLNSDGPYLLHVSIDELENVWPLVPPGASNSEMLEKLS

SEQ ID NO: 13 - DNA, 2111 bp, Escherichia coli; identical to SEQ ID NO: 11
  RBS (8)..(12)

SEQ ID NO: 14 - protein, 87 aa, Escherichia coli
MMQHQVNVSARFNPETLERVLRVVRHRGFHVCSMNMAAASDAQNINIELTVASPRSVDLLFSQLNKLVDVAHVAICQSTTTSQQIRA

(19) United States
(12) Patent Application Publication
(10) Pub. No.: US 2003/0073204 A1
(43) Pub. Date: Apr. 17, 2003

(54) Process for the manufacture of D-pantothene acid and/or its salts by fermentation

(75) Inventor: Mechthild Rieping, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Marl (DE)

(21) Appl. No.: 10/096,595

(22) Filed: Mar. 14, 2002

Related U.S. Application Data
(60) Provisional application No. 60/304,774, filed on Jul. 13, 2001.

(30) Foreign Application Priority Data
Mar. 14, 2001 (DE) 101 12 100.8

(51) Int. Cl. C12P 13/04; C12N 1/21

(52) U.S. Cl. 435/106; 435/252.33

(57) ABSTRACT

The invention provides a process for the preparation of D-pantothenic acid and/or salts thereof or feedstuffs additives comprising these by fermentation of microorganisms of the Enterobacteriaceae family, in particular those which already produce D-pantothenic acid, characterized in that the nucleotide sequence(s) in the microorganisms which code(s) for the pckA gene is (are) attenuated, in particular eliminated.

### CROSS-REFERENCE TO RELATED APPLICATIONS

This application claims priority to DE 101 12 100.8, filed Mar. 14, 2001 and to U.S. provisional application 60/304,774, filed Jul. 13, 2001. The entire contents of both documents are incorporated by reference.

### REFERENCE TO SEQUENCE LISTING

The contents of the Sequence Listing in computer readable form as provided herewith are hereby incorporated by reference.

### BACKGROUND OF THE INVENTION

1. Field of the Invention

An improved process for the fermentive preparation of D-pantothenic acid and its derivatives using a microorganism in which the pckA gene is attenuated or eliminated, especially microorganisms of the Enterobacteriaceae family. Nucleic acids and vectors encoding an attenuated pckA gene, and host cells comprising such nucleic acids or vectors, or in which the pckA gene has been eliminated.

2. Description of Related Art

World-wide production of pantothenic acid is the order of magnitude of several thousand tons a year. Pantothenic acid has a variety of uses, including in medical, pharmaceutical and nutritional products, including foodstuffs or feedstuffs. Additionally, a significant portion of the pantothenic acid produced is used for the nutrition of stock animals such as poultry and pigs.

While pantothenic acid can be prepared by chemical synthesis, chemical synthesis results in the production of a racemic mixture of DL-pantothenic acid, which must be further purified to obtain the naturally occurring, more biologically active D-pantothenic acid. DL-pantolactone is an important precursor of DL-pantothenic acid in the chemical synthesis of DL-pantothenic acid and may be prepared in a multi-stage process from formaldehyde, isobutylaldehyde and cyanide. The resulting racemic mixture of DL-pantolactone is subsequently separated and subjected to a condensation reaction with β-alanine. D-pantothenic acid is subsequently obtained.

Pantothenic acid may also be prepared by fermentation using a suitable microorganism. One advantage of the fermentative preparation by microorganisms lies in the direct formation of the desired stereoisomeric form, that is to say the D-form, which is free from L-pantothenic acid.

D-pantothenic acid can be produced by various types of bacteria, such as Escherichia coli (E. coli), Arthrobacter ureafaciens, Corynebacterium erythrogenes, Brevibacterium ammoniagenes, and also by yeasts, such as Debaromyces castellii. Typical nutrient media or solutions comprise glucose, DL-pantoic acid and β-alanine, as shown in EP-A 0 493 060. EP-A 0 493 060 also shows that in E. coli the formation of D-pantothenic acid is improved by the amplification of E. coli pantothenic acid biosynthesis genes contained on plasmids pFV3 and pFV5, and use of a nutrient solution comprising glucose, DL-pantoic acid and β-alanine.

EP-A 0 590 857 and U.S. Pat. No. 5,518,906 describe mutants derived from E. coli strain IF03547, such as FV5714, FV525, FV814, FV521, FV221, FV6OSl and FV5069, which carry resistances to various antimetabolites, such as salicylic acid, α-ketobutyric acid, β-hydroxyaspartic acid, O-methylthreonine and α-ketoisovaleric acid. Such strains or mutants produce pantoic acid in a nutrient solution comprising glucose, and produce D-pantothenic acid in a nutrient solution comprising glucose and β-alanine.

EP-A 0 590 857 and U.S. Pat. No. 5,518,906 indicate that after amplification of the pantothenic acid biosynthesis genes panB, panC and panD, which are said to be contained on the plasmid pFV31, in the above-mentioned strains the production of D-pantoic acid in nutrient solutions comprising glucose and the production of D-pantothenic acid in a nutrient solution comprising glucose and β-alanine is improved.

The favorable effect of enhancement of the ilvGM operon on production of D-pantothenic acid is also reported in WO97/10340. Finally, the effect of enhancement of the panE gene on the formation of D-pantothenic acid is reported in EP-A-1001027.

D-pantothenic acid or the corresponding salt may be isolated from the fermentation broth and purified (EP-A-0590857 and WO96/33283) and subsequently used in purified form, or alternatively, the fermentation broth comprising D-pantothenic acid may be dried (EP-A-1050219) and used in particular as a feedstuffs additive.

In view of the importance of D-pantothenic acid, its would be highly desirable to have a more simple, economical and efficient process that produces high yields of D-pantothenic acid or its derivatives.

### BRIEF SUMMARY OF THE INVENTION

The present invention provides a more simple, improved, economical and efficient process for producing D-pantothenic acid by fermentive production using a microorganism selected or modified to contain an attenuated pckA gene or a microorganism in which the pckA gene has been eliminated.

Another object of the present invention is a process that provides high yields of D-pantothenic acid useful in pharmaceutical products, foods, nutritional products or animal feeds.

An additional object of the invention is to provide a derivative of pantothenic acid, such as a calcium salt, that is more stable or easily handled or processed that D-pantothenic acid (free acid).

Yet another object of the invention is provision of improved animal feedstocks or feedstock additives containing high amounts of D-pantothenic acid, as well as feedstocks or feedstock additives in which the content of D-pantothenic acid or its derivative is more stable or biologically available. Thus, for example, the invention provides a process in which, after conclusion of the fermentation, all or some of the biomass remains in the fermentation broth, and the broth obtained in this way is processed, optionally after concentration, to a solid mixture which comprises D-pantothenic acid and/or salts thereof and also comprises further constituents of the fermentation broth.

The invention also includes the formulation or supplementation of products, such as pharmaceuticals, nutritional products, cosmetics, foods, or feedstuffs using either the isolated and purified D-pantothenic acid or one or more of its derivatives, or alternatively, the biomass or dried and/or concentrated fermentation broth or culture medium.

Other objects of the invention include nucleic acids and vectors that encode attenuated pckA genes, as well as cells, such as E. coli, containing such genes or vectors.

### BRIEF DESCRIPTION OF THE DRAWINGS (FIGURES)

FIG. 1: pMAK705ΔpckA (=pMAK705deltapckA)

FIG. 2: pTrc99AilvGM

FIG. 3: pFV31ilvGM

The length data are to be understood as approx. data. The abbreviations and designations used have the following meaning:

cat: Chloramphenicol resistance gene

rep-ts: Temperature-sensitive replication region of the plasmid pSC101

pck1: Part of the 5′ region of the pckA gene

pck2: Part of the 3′ region of the pckA gene

Amp: Ampicillin resistance gene

lacI: Gene for the repressor protein of the trc promoter

Ptrc: trc promoter region, IPTG-inducible

ilvG: Coding region of the large subunit of acetohydroxy acid synthase II

ilvM: Coding region of the small subunit of acetohydroxy acid synthase II

5S: 5S rRNA region

rrnBT: rRNA terminator region

panB: Coding region of the panB gene

panC: Coding region of the panC gene

The abbreviations for the restriction enzymes have the following meaning

BamHI: Restriction endonuclease from Bacillus amyloliquefaciens

BglII: Restriction endonuclease from Bacillus globigii

ClaI: Restriction endonuclease from Caryphanon latum

EcoRI: Restriction endonuclease from Escherichia coli

EcoRV: Restriction endonuclease from Escherichia coli

HindIII: Restriction endonuclease from Haemophilus influenzae

KpnI: Restriction endonuclease from Klebsiella pneumoniae

PstI: Restriction endonuclease from Providencia stuartii

PvuI: Restriction endonuclease from Proteus vulgaris

SacI: Restriction endonuclease from Streptomyces achromogenes

SalI: Restriction endonuclease from Streptomyces albus

SmaI: Restriction endonuclease from Serratia marcescens

SphI: Restriction endonuclease from Streptomyces phaeochromogenes

SspI: Restriction endonuclease from Sphaerotilus species

XbaI: Restriction endonuclease from Xanthomonas badrii

XhoI: Restriction endonuclease from Xanthomonas holcicola

### DETAILED DESCRIPTION OF THE INVENTION

The terms “D-pantothenic acid”, “pantothenic acid” or “pantothenate” as used in this disclosure encompass both the free acids and salts of D-pantothenic acid, such as the calcium, sodium, ammonium or potassium salts. Pantothenic acid derivatives, such as the alcohol, aldehyde, alcohol esters or acid esters, which may be bioconverted or metabolized into pantothenic acid once ingested or administered to a living organism, such as a mammal, are also contemplated.

The term “pckA gene” is known in the art. This gene codes for phosphoenol pyruvate carboxylase (EC 4.1.1.49). An exemplary pckA gene is that of Escherichia coli. The nucleotide sequence of the pckA gene of Escherichia coli has been published by Medina et al., Journal of Bacteriology 172, 7151-7156 (1990) and can also be found in the genome sequence of Escherichia coli published by Blattner et al., Science 277, 1453 - 1462 (1997) under Accession Number AE000416. This term is also intended to include various allelic forms of this gene as well as modified versions of this gene as described below.

The term “attenuated microorganism” refers to one in which the amount or activity of one or more enzymes or other biologically active proteins is reduced or eliminated. For instance, a microorganism with an attenuated pckA gene may either be deleted for the pckA gene, carry a modified form of the pckA gene that encodes an enzyme with lowered activity, or may carry a pckA gene with regulatory sequences that decrease its expression. The term “attenuation” in this connection describes the reduction or elimination of the intracellular activity of one or more enzymes (proteins) in a microorganism which are coded by the corresponding DNA, for example, by using a weak promoter or using a gene or allele which codes for a corresponding enzyme (protein) with a low activity or inactivates the corresponding gene or enzyme (protein), or optionally combining these measures.

By attenuation measures, including reduction or elimination of expression, the activity or concentration of the corresponding protein is reduced by at least 0-75%, 0-50%, 0 to 25%, 0 to 10%, 0 to 5%, or 0 to 1% of the activity or concentration of the wild-type protein or of the activity or concentration of the protein in the starting microorganism.

The improved process of the present invention produces D-pantothenic acid or it salts or derivatives using a microorganism in which the pckA gene has been attenuated or deleted. Advantageously, a microorganism of the Enterobacteriaceae family is used, for instance, an E. coli strain. Advantageously, a microorganism already known to produce D-pantothenic acid can be modified to attenuate or eliminate the pckA gene.

Advantageously, the inventive process may be characterized by the following:

a) Fermentation of a microorganism of the Enterobacteriaceae family in which the pckA gene is attenuated (or eliminated) in a culture medium suitable for the production of D-pantothenic acid. Optionally, the microorganism used may contain other attenuated or enhanced genes that improve the yield, stability or efficiency of production of D-pantothenic acid or its derivatives.

b) Fermentation may optionally be conducted in the presence of one or more alkaline earth metal compound(s), which may be added continuously or discontinuously to the culture medium preferably in a stoichiometric amount.

c) Concentration of the D-pantothenic acid or its corresponding salt or other derivative produced by the fermentation process may be accomplished by further processing of the culture medium, fermentation broth or microorganisms used in the fermentation process.

d) Upon conclusion of the fermentation, the D-pantothenic acid, or its corresponding salt or derivative, may be further purified or isolated.

Optionally, further modification or derivativization, such as esterification of the panthothenic acid or its salt may be conducted, for instance, to improve stability, handling properties or absorption of this compound. Such modifications may be made either as part of the fermenation process or after concentration, isolation or purification of D-pantothenic acid or its salt. For instance, D-pantothenic acid may be further converted into a more stable derivative, such as the alcohol derivative pantothenol.

The microorganisms which the present invention provides can produce D-pantothenic acid from a variety of carbohydrates, sugars or other carbon-containing substrates, including glucose, sucrose, lactose, fructose, maltose, molasses, starch, cellulose or from glycerol and ethanol.

These strains are representatives of Enterobacteriaceae, in particular of the genus Escherichia. Advantageously, from the genus Escherichia, the species Escherichia coli is to be mentioned in particular. Within the species Escherichia coli there may be mentioned the so-called K-12 strains, such as e.g. the strains MG1655 or W3110 (Neidhard et al.: Escherichia coli and Salmonella. Cellular and Molecular Biology (ASM Press, Washington D.C.)) or the Escherichia coli wild type strain IFO3547 (Institute of Fermentation, Osaka, Japan) and mutants derived from these, which have the ability to produce D-pantothenic acid.

Suitable D-pantothenic acid-producing strains of the genus Escherichia, in particular of the species Escherichia coli, are, for example

Escherichia coli FV5069/pFV31

Escherichia coli FV5069/pFV202

Escherichia coli FE6/pFE80 and

Escherichia coli KE3

It has been found that Enterobacteriaceae produce D-pantothenic acid in an improved manner after attenuation of the pckA gene, which codes for phosphoenol pyruvate carboxykinase (EC 4.1.1.49). However, the present invention may use any suitable pckA gene, including those described in the text references mentioned above. Alleles of the pckA gene, which result from the degeneracy of the genetic code or due to sense mutations of neutral function, can also be used. Moreover, nucleic acid sequences which cross-hybridize to the pckA genes described above under stringent conditions and that encode polypeptides having phospholenol pyruvate activity may also be used, for instance, a nucleic acid sequence that (a) encodes a pckA gene product with reduced enzymatic activity compared to the gene product encoded by SEQ ID NO: 1 and (b) that is at least 70%, 80%, 90%, 95% or 99% similar to that of SEQ ID NO: 1 or that hybridizes with SEQ ID NO: 1 under stringent conditions, wherein stringent conditions comprise washing in 5×SSC at a temperature ranging from 50° to 68° C.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Attenuation may be achieved by reducing the expression of the pckA gene or by reducing or eliminating the catalytic properties of the enzyme it encodes. These measures may also be combined.

The reduction in gene expression can take place by suitable culturing, by genetic modification (mutation) of the signal structures of gene expression or also by the antisense-RNA technique. Signal structures of gene expression are, for example, repressor genes, activator genes, operators, promoters, attenuators, ribosome binding sites, the start codon and terminators. Methods for reducing or enhancing gene expression are known in the art, however, pertinent information may be also found, inter alia, in Jensen and Hammer, Biotechnology and Bioengineering 58: 191-195 (1998), in Carrier and Keasling, Biotechnology Progress 15, 58-64 (1999), Franch and Gerdes, Current Opinion in Microbiology 3, 159-164 (2000) and in known textbooks of genetics and molecular biology, such as, for example, the textbook of Knippers, “Molekulare Genetik [Molecular Genetics]”, 6th edition, Georg Thieme Verlag, Stuttgart, Germany, (1995) or that of Winnacker, “Gene und Klone [Genes and Clones]”, VCH Verlagsgesellschaft, Weinheim, Germany, (1990).

Certain mutations, which lead to a change or reduction in the catalytic properties of enzyme proteins, are known from the prior art. Examples which may be mentioned are the works of Qiu and Goodman, Journal of Biological Chemistry 272: 8611-8617 (1997), Yano et al., Proceedings of the National Academy of Sciences, USA 95, 5511-5515 (1998), Wente and Schachmann, Journal of Biological Chemistry 266, 20833-20839 (1991). Summarizing descriptions can be found in known textbooks of genetics and molecular biology, such as e.g. that by Hagemann, “Allgemeine Genetik [General Genetics]”, Gustav Fischer Verlag, Stuttgart, (1986).

Possible mutations include transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, which lead to incorrect amino acids being incorporated or translation being interrupted prematurely. Deletions of several codons typically lead to a complete loss of the enzyme activity. Instructions on generation of such mutations are prior art and can be found in known textbooks of genetics and molecular biology, such as e.g. the textbook by Knippers, “Molekulare Genetik [Molecular Genetics]”, 6th edition, Georg Thieme Verlag, Stuttgart, Germany, (1995), that by Winnacker, “Gene und Klone [Genes and Clones]”, VCH Verlagsgesellschaft, Weinheim, Germany, (1990) or that by Hagemann, “Allgemeine Genetik [General Genetics]”, Gustav Fischer Verlag, Stuttgart, (1986).

Suitable mutations in the pckA gene, such as, for example, deletion mutations, can be incorporated into suitable strains by gene or allele replacement.

A conventional method is the method, described by Hamilton et al., Journal of Bacteriology 174, 4617-4622 (1989), of gene replacement with the aid of a conditionally replicating pSC101 derivative pMAK705. Other methods described in the prior art, such as, for example, those of Martinez-Morales et al., Journal of Bacteriology 1999: 7143-7148 (1999) or those of Boyd et al., Journal of Bacteriology 182, 842-847 (2000), can likewise be used.

It is also possible to transfer mutations in the pckA gene or mutations that affect expression of the pckA gene into various strains by conjugation or transduction.

In addition to the attenuation of the pckA gene to increase the amount, simplicity or efficiency of the production of D-pantothenic acid, other enhanced genes may be added to a microbial strain, such as a strain of the Enterobacteriaceae family. For instance, one or more of the following genes may be enhanced or over-expressed in an appropriate microbial strain:

the ilvGM operon which codes for acetohydroxy-acid synthase II (WO 97/10340)

the panB gene which codes for ketopantoate hydroxymethyl transferase (U.S. Pat. No. 5,518,906),

the panE gene which codes for ketopantoate reductase (EP-A-1001027)

the panD gene which codes for aspartate decarboxylase (U.S. Pat. No. 5,518,906)

the panC gene which codes for pantothenate synthetase (U.S. Pat. No. 5,518,906)

the serC gene which codes for phosphoserine transaminase (Duncan and Coggins, Biochemical Journal 234:49-57 (1986)),

the gcvT, gcvH and gcvP genes which code for the glycine cleavage system (Okamura-Ikeda et al., European Journal of Biochemistry 216, 539-548 (1993)), and

the glyA gene which codes for serine hydroxymethyl transferase (Plamann et al., Nucleic Acids Research 11(7):2065-2075(1983)).

The term “enhancement” in this connection describes the increase in the intracellular activity of one or more enzymes or proteins in a microorganism which are encoded by the corresponding DNA. Such enhancement may be achieved, for example, by increasing the number of copies of the gene or genes, using a potent promoter or a gene or allele which codes for a corresponding enzyme or protein with a high activity, or optionally combining these measures.

By enhancement measures, in particular over-expression, the activity or concentration of the corresponding protein is in general increased by at least 1%, 5%, 10%, 25%, 50%, 75%, 100%, 150%, 200%, 300%, 400% or 500%, including 1000% or 2000%, based on that of the wild-type protein or the activity or concentration of the protein in the starting microorganism.

Finally, it may be advantageous for the production of D-pantothenic acid with microbial stains, such as strains of the Enterobacteriaceae family, in addition to the attenuation of the pckA gene, for particular genes to be attenuated, eliminated or expressed at a low level. Such genes include:

the avtA gene which codes for transaminase C (EP-A-1001027) and

the poxB gene which codes for pyruvate oxidase (Grabau and Cronan, Nucleic Acids Research. 14 (13), 5449-5460 (1986)).

In addition to the attenuation of the pckA gene it may furthermore be advantageous for the production of D-pantothenic acid to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982). Microbes, such as bacteria in which the metabolic pathways that reduce the formation of D-pantothenic acid are at least partly eliminated, can be employed in the process according to the invention.

Any suitable cultivation method may be selected. For instance, the microorganisms produced according to the invention can be cultured in the batch process (batch culture), the fed batch (feed process) or the repeated fed batch process (repetitive feed process). A summary of known culture methods is described in the textbook by Chmiel (Bioprozesstechnik 1. Einführung in die Bioverfahrenstechnik [Bioprocess Technology 1. Introduction to Bioprocess Technology, Gustav Fischer Verlag, Stuttgart, (1991) or in the textbook by Storhas, Bioreaktoren und periphere Einrichtungen [Bioreactors and Peripheral Equipment] (Vieweg Verlag, Braunschweig/Wiesbaden (1994).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA (1981). Sugars and carbohydrates, such as e.g. glucose, sucrose, lactose, fructose, maltose, molasses, starch and cellulose, oils and fats, such as soya oil, sunflower oil, groundnut oil and coconut fat, fatty acids, such as palmitic acid, stearic acid and linoleic acid, alcohols, such as glycerol and ethanol, and organic acids, such as acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus.

The culture medium must furthermore comprise salts of metals, such as magnesium sulfate or iron sulfate, which are necessary for growth. Finally, essential growth substances, such as amino acids and vitamins, can be employed in addition to the above-mentioned substances. Precursors of pantothenic acid, such as aspartate, β-alanine, ketoisovalerate, ketopantoic acid or pantoic acid and optionally salts thereof, can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture.

For the preparation of an alkaline earth metal salt of pantothenic acid, in particular the calcium salt, it is equally possible to add the suspension or solution of an inorganic compound containing an alkaline earth metal, such as, for example, calcium hydroxide, or of an organic compound, such as the alkaline earth metal salt of an organic acid, for example calcium acetate, continuously or discontinuously during the fermentation. In this manner, the cation necessary for preparation of the desired alkaline earth metal salt of D-pantothenic acid is introduced into the fermentation broth directly in the desired amount, preferably in stoichiometric amounts.

Antifoams, such as fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of D-pantothenic acid has formed. This target is usually reached within 10 hours to 160 hours.

The D-pantothenic acid or the corresponding salts of D-pantothenic acid contained in the fermentation broth can be isolated and purified in accordance with known methods.

It is also possible for the fermentation broths comprising D-pantothenic acid and/or salts thereof preferably first to be freed from all or some of the biomass by known separation methods, such as, for example, centrifugation, filtration, decanting or a combination thereof. However, it is also possible to leave the biomass in its entirety in the fermentation broth. In general, the suspension or solution is preferably concentrated and worked up to a powder, for example with the aid of a spray dryer or a freeze-drying unit. This powder is then in general converted by suitable compacting or granulating processes, e.g. also build-up granulation, into a coarser-grained, free-flowing, storable and largely dust-free product with the desired particle size distribution of optionally 20 to 2000 μm. In the granulation or compacting it is advantageous to employ conventional organic or inorganic auxiliary substances or carriers, such as starch, gelatin, cellulose derivatives or similar substances, such as are conventionally used as binders, gelling agents or thickeners in foodstuffs or feedstuffs processing, or further substances, such as, for example, silicas, silicates or stearates.

Alternatively, the fermentation product, with or without further conventional fermentation constituents, can be absorbed on to an organic or inorganic carrier substance which is known and conventional in feedstuffs processing, such as, for example, silicas, silicates, grits, brans, meals, starches, sugars or others, and/or stabilized with conventional thickeners or binders. Use examples and processes in this context are described in the literature (Die Mühle+Mischfuttertechnik 132 (1995) 49, page 817). D-Pantothenic acid, or the desired salt of D-pantothenic acid or a formulation comprising these compounds, is optionally added at a suitable process stage in order to achieve or establish the desired content of pantothenic acid, pantothenic acid derivative, or of the desired salt in the end product.

The desired content is in general in the range from 20 to 80 wt. % (dry mass).

The concentration of pantothenic acid can be determined with known chemical (Velisek; Chromatographic Science 60, 515-560 (1992)) or microbiological methods, such as e.g. the Lactobacillus plantarum test (DIFCO MANUAL, 10^{th} Edition, p. 1100-1102; Michigan, USA).

Products, such as feedstuffs or feedstuff additives, obtained by the concentration of the culture medium, fermentation medium, or fermentive microorganisms may be further supplemented with other nutritional products, such as minerals or vitamins. For instance, pantothenic acid synergists such as vitamin B12 may be added. Other substances that facilitate the uptake or utilization of pantothenic acid, such as beta-carotene, vitamin A, vitamin C, vitamin E, vitamin B6, folic acid or biotin may also be added. Moreover, the pH of such products may be adjusted using convention acids, bases or buffers to enhance the stability of the pantothenic acid, its salt or derivative.

Such feedstuffs or feedstuff additives may be administered to livestock animals, such as bovines, pigs, goats, sheep, horses, camels, and llamas; avians, such as chickens, geese or ducks; pets or domestic animals, such as cat and dogs, birds or tropical fish; laboratory animals, such as mice, rats and hamsters; wild herbiferous, omnivorous or carnivorous animals such reptiles, zebras, elephants, bears, lions and tigers; or may be added to aquaculture fees, such as those used for fish or crustaceans; or to products used to feed insects, such as bees.

A pure culture of the Escherichia coli K-12 strain DH5α/pMAK705 was deposited as DSM 13720 on 8^{th} September 2000 at the Deutsche Sammlung fur Mikroorganismen und Zellkulturen (DSMZ=German Collection of Microorganisms and Cell Cultures, Braunschweig, Germany) in accordance with the Budapest Treaty.

A pure culture of the Escherichia coli K-12 strain MG442ΔpckA was deposited as DSM 13761 on Oct. 2, 2000 at the Deutsche Sammlung für Mikroorganismen und Zellkulturen (DSMZ=German Collection of Microorganisms and Cell Cultures, Braunschweig, Germany) in accordance with the Budapest Treaty.

The present invention is explained in more detail in the following with the aid of embodiment examples.

The isolation of plasmid DNA from Escherichia coli and all techniques of restriction, Klenow and alkaline phosphatase treatment are carried out by the method of Sambrook et al. (Molecular cloning—A Laboratory Manual, Cold Spring Harbor Laboratory Press (1989). Unless described otherwise, the transformation of Escherichia coli is carried out by the method of Chung et al., Proceedings of the National Academy of Sciences of the United States of America USA 86: 2172-2175 (1989).

The incubation temperature for the preparation of strains and transformants is 37° C. Temperatures of 30° C. and 44° C. are used in the gene replacement method of Hamilton et.al. ibid. (1989).

### EXAMPLE 1

Construction of the deletion mutation of the pckA gene

Parts of the 5′ and 3′ region of the pckA gene are amplified from Escherichia coli K12 using the polymerase chain reaction (PCR) and synthetic oligonucleotides. Starting from the nucleotide sequence of the pckA gene in

E. coli K12 MG1655 (SEQ ID No. 1), the following PCR primers are synthesized (MWG Biotech, Ebersberg, Germany):

pckA′5′-1: 5′- GATCCGAGCCTGACAGGTTA -3′ (SEQ ID No. 3)

pckA′5′-2: 5′- GCATGCGCTCGGTCAGGTTA -3′ (SEQ ID No. 4)

pckA′3′-1: 5′- AGGCCTGAAGATGGCACTATCG -3′ (SEQ ID No. 5)

pckA′3′-2: 5′- CCGGAGAAGCGTAGGTGTTA -3′ (SEQ ID No. 6)

The chromosomal E. coli K12 MG1655 DNA employed for the PCR is isolated according to the manufacturers instructions with “Qiagen Genomic-tips 100/G” (QIAGEN, Hilden, Germany). A DNA fragment approx. 500 bp in size from the 5′ region of the pckA gene (called pck1) and a DNA fragment approx. 600 bp in size from the 3′ region of the pckA gene (called pck2) can be amplified with the specific primers under standard PCR conditions (Innis et al. (1990) PCR Protocols. A Guide to Methods and Applications, Academic Press) with Taq-DNA polymerase (Gibco-BRL, Eggenstein, Germany). The PCR products are each ligated with the vector pCR2.1TOPO (TOPO TA Cloning Kit, Invitrogen, Groningen, The Netherlands) in accordance with the manufacturers instructions and transformed into the E. coli strain TOP10F′.

Selection of plasmid-carrying cells takes place on LB agar, to which 50 μg/ml ampicillin are added. After isolation of the plasmid DNA, the vector pCR2.1TOPOpck2 is cleaved with the restriction enzymes StuI and XbaI and, after separation in 0.8% agarose gel, the pck2 fragment is isolated with the aid of the QIAquick Gel Extraction Kit (QIAGEN, Hilden, Germany). After isolation of the plasmid DNA the vector pCR2.1TOPOpck1 is cleaved with the enzymes EcoRV and XbaI and ligated with the pck2 fragment isolated. The E. coli strain DH5α is transformed with the ligation batch and plasmid-carrying cells are selected on LB agar, to which 50 μg/ml ampicillin is added. After isolation of the plasmid DNA those plasmids in which the mutagenic DNA sequence shown in SEQ ID No. 7 is cloned are detected by control cleavage with the enzymes SpeI and XbaI. One of the plasmids is called pCR2.1TOPOΔpckA.

### EXAMPLE 2

Construction of the replacement vector pMAK705ΔpckA

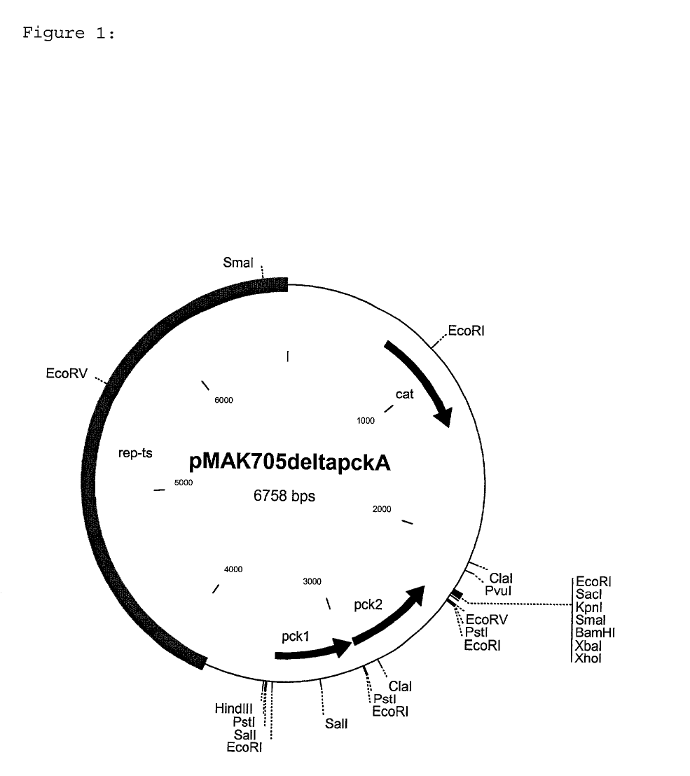

The pckA allele described in example 1 is isolated from the vector pCR2.1TOPOΔpckA after restriction with the enzymes SpeI and XbaI and separation in 0.8% agarose gel, and ligated with the plasmid pMAK705 (Hamilton et al. (1989) Journal of Bacteriology 174, 4617-4622), which has been digested with the enzyme XbaI. The ligation batch is transformed in DH5α and plasmid-carrying cells are selected on LB agar, to which 20 μg/ml chloramphenicol are added. Successful cloning is demonstrated after isolation of the plasmid DNA and cleavage with the enzymes HpaI, KpnI, HindIII, SalI and PstI. The replacement vector formed, pMAK705ΔpckA (=pMAK705deltapckA), is shown in FIG. 1.

### EXAMPLE 3

Position-specific mutagenesis of the pckA gene in the E. coli strain MG442

The L-threonine-producing E. coli strain MG442 is described in the patent specification U.S. Pat. No. 4,278,765 and deposited as CMIM B-1628 at the Russian National Collection for Industrial Microorganisms (VKPM, Moscow, Russia).

For replacement of the chromosomal pckA gene with the plasmid-coded deletion construct, MG442 is transformed with the plasmid pMAK705ΔpckA, The gene replacement is carried out by the selection method described by Hamilton et al. (1989) Journal of Bacteriology 174, 4617-4622) and is verified by standard PCR methods (Innis et al. (1990) PCR Protocols. A Guide to Methods and Applications, Academic Press) with the following oligonucleotide primers:

pckA′5′-1: 5′- GATCCGAGCCTGACAGGTTA -3′ (SEQ ID No. 3)

pckA′3′-2: 5′- CCGGAGAAGCGTAGGTGTTA -3′ (SEQ ID No. 6)

After replacement has taken place, MG442 contains the form of the ΔpckA allele shown in SEQ ID No. 8. The strain obtained is called MG442ΔpckA.

### EXAMPLE 4

Preparation of D-pantothenic acid with the strain MG442ΔpckA/pFV3lilvGM

4.1 Amplification and Cloning of the ilvGM Gene

The ilvGM operon from Escherichia coli IF03547 which codes for acetohydroxy acid synthase II (Institut für Fermentation [Institute of Fermentation], Osaka, Japan) is amplified using the polymerase chain reaction (PCR) and synthetic oligonucleotides. Starting from the nucleotide sequence of the ilvGM operon in E. coli K12 MG1655 (GenBank: Accession No. M87049), PCR primers are synthesized, (MWG Biotech, Ebersberg, Germany). The sequence of the primer ilvGM1 is chosen such that it contains an adenine at position 8. As a result, a modified ribosome binding site is generated 7 nucleotides upstream of the start codon of the ilvG protein.

IlvGM1: 5′-CAGGACGAGGAACTAACTATG -3′ (SEQ ID No. 9)

IlvGM2: 5′-TCACGATGGCGGAATACAAC -3′ (SEQ ID No. 10)

The chromosomal E. coli IF03547 DNA employed for the PCR is isolated according to the manufacturers instructions with “QIAGEN Genomic-tips 100/G” (QIAGEN, Hilden, Germany). A DNA fragment approx. 2100 bp in size, which comprises the modified ribosome binding site, the ilvGM coding regions and approx. 180 bp 3′-flanking sequences, can be amplified with the specific primers under standard PCR conditions (Innis et al.: PCR Protocols. A Guide to Methods and Applications, 1990, Academic Press) with Pfu-DNA polymerase (Promega Corporation, Madison, USA). The PCR product is cloned in the plasmid pCR-BluntII-TOPO and transformed in the E. coli strain TOP10 (Invitrogen, Groningen, The Netherlands, Product Description Zero Blunt TOPO PCR Cloning Kit, Cat. No. K2800-20). Successful cloning is demonstrated by cleavage of the plasmid pCR-BluntIFO3547ilvGM with the restriction enzymes EcoRI and SphI. For this, the plasmid DNA is isolated by means of the “QIAprep Spin Plasmid Kit” (QIAGEN, Hilden, Germany) and, after cleavage, separated in a 0.8% agarose gel. The DNA sequence of the amplified fragment is determined using the reverse and universal sequencing primer (QIAGEN, Hilden, Germany). The sequence of the PCR product is shown in SEQ ID No. 11 and 13. The ilvG gene or allele is identified in SEQ ID No. 11. The ilvM gene or allele is identified in SEQ ID No. 13. The associated gene products or proteins are shown in SEQ ID No. 12 and 14.

4.2 Cloning of the ilvGM Gene in the Expression Vector pTrc99A

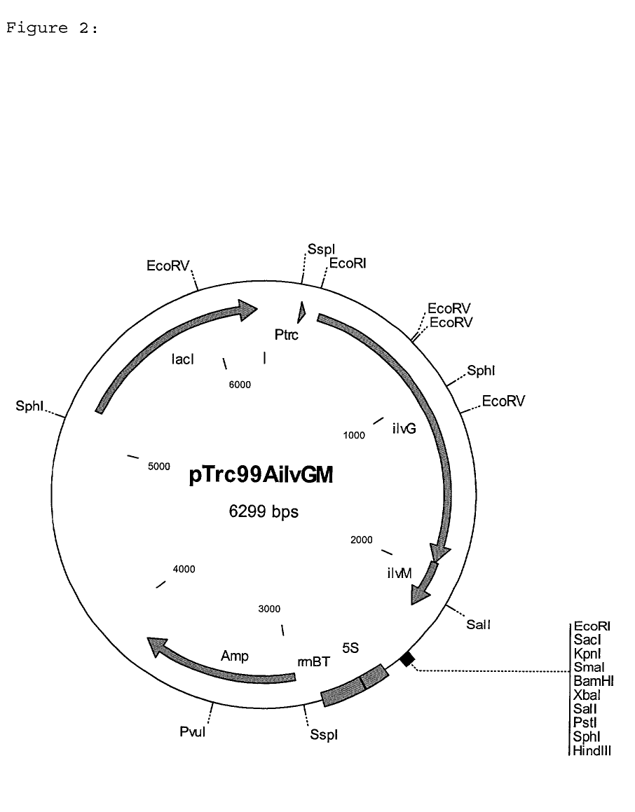

The ilvGM genes described in example 4.1 are cloned in the vector pTrc99A (Amersham Pharmacia Biotech Inc, Uppsala, Sweden) for expression in Escherichia coli K12. For this, the plasmid pCR-BluntIFO3547ilvGM is cleaved with the enzyme EcoRI, the cleavage batch is separated in 0.8% agarose gel and the ilvGM fragment 2.1 kbp in size is isolated with the aid of the QIAquick Gel Extraction Kit (QIAGEN, Hilden, Germany). The vector pTrc99A is cleaved with the enzyme EcoRI, an alkaline phosphatase treatment is carried out, and ligation is carried out with the ilvGM fragment isolated. The ligation batch is transformed in the E. coli strain DH5A. Selection of pTrc99A-carrying cells is carried out on LB agar (Lennox, Virology 1:190 (1955)), to which 50 μg/ml ampicillin is added. Successful cloning of the ilvGM operon can be demonstrated after plasmid DNA isolation and control cleavage with SalI and SphI. In the vector, which is called pTrc99AilvGM (FIG. 2), expression of the ilvGM operon is regulated by the Ptrc promoter lying upstream of the modified ribosome binding site and by the rRNA terminator region lying downstream of the ilvGM coding region.

4.3 Construction of the Vector pFV31ilvGM

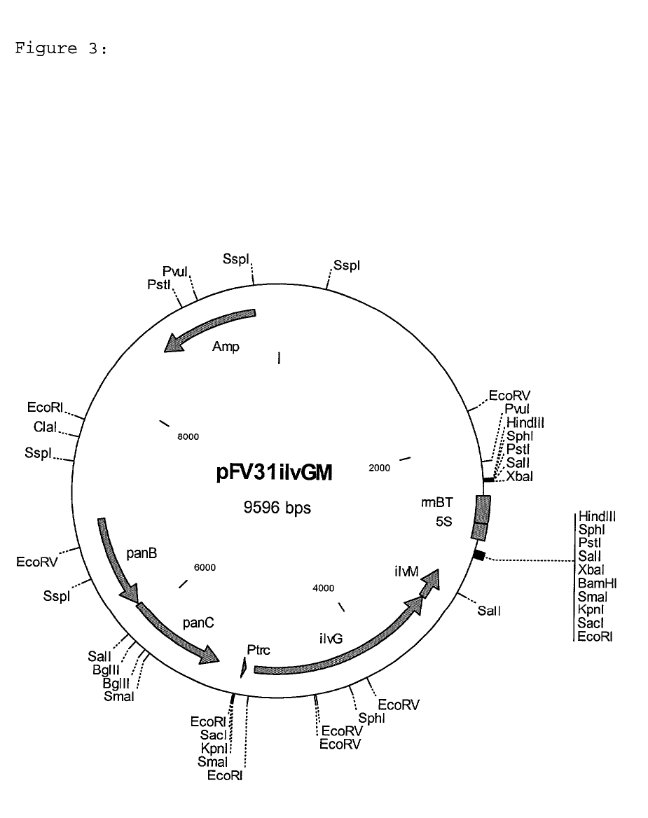

The E. coli strain FV5069/pFV31 which produces D-pantothenic acid is described in EP-A-0590857 and deposited as FERM BP 4395 in accordance with the Budapest Treaty. The plasmid pFV31 is isolated from FV5069/pFV31, cleaved with the enzyme BamHI, and the projecting 3′ ends are treated with Klenow enzyme. An alkaline phosphatase treatment is then carried out. From the vector pTrc99AilvGM described in example 4.2, after restriction with the enzyme SspI and separation of the cleavage batch in 0.8% agarose gel, the ilvGM expression cassette 2.8 kbp in size is isolated and ligated with the linearized and dephosphorylated vector pFV31. The ligation batch is transformed in the E. coli strain DH5α and plasmid-carrying cells are selected on LB agar, to which 50 μg/ml ampicillin are added. Successful cloning of the ilvGM expression cassette can be demonstrated after plasmid DNA isolation and control cleavage with HindIII, SalI, SmaI, SphI and XbaI. The plasmid is called pFV31ilvGM (FIG. 3).

4.4 Preparation of the Strain MG442ΔpckA/pFV3lilvGM

The strain MG442ΔpckA obtained in example 3 and the strain MG442 are transformed with the plasmid pFV31ilvGM and transformants are selected on LB agar, which is supplemented with 50 μg/ml ampicillin. The strains MG442ΔpckA/pFV31ilvGM and MG442/pFV31ilvGM are formed in this manner.

4.5 Preparation of D-pantothenic Acid with the Strain MG442ΔpckA/pFV31ilvGM

The pantothenate production of the E. coli strains MG442/pFV31ilvGM and MG442ΔpckA/pFV31ilvGM is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20/1 glucose, 50 μg/ml ampicillin, are inoculated with an individual colony and incubated for 20 hours at 33° C. and 200 rpm on an ESR incubator from Kühner AG (Birsfelden, Switzerland). In each case 200 μl of this preculture are transinoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose, 20 g/l β-alanine, 250 mg/l thiamine) and the batch is incubated for 48 hours at 37° C. and 200 rpm. After the incubation the optical density (OD) of the culture suspension is determined with an LP2W photometer from Dr. Lange (Düsseldorf, Germany) at a measurement wavelength of 660 nm.

The concentration of D-pantothenate formed in the sterile-filtered culture supernatant is then determined by means of the Lactobacillus plantarum ATCC8014 pantothenate assay in accordance with the instructions of DIFCO, DIFCO MANUAL, 10^{th} Edition, p. 1100-1102; Michigan, USA. D(+)-Pantothenic acid calcium salt hydrate (catalogue number 25,972-1, Sigma-Aldrich, Deisenhofen, Germany) is used for the calibration.

The result of the experiment is shown in table 1.

TABLE 1

| | OD | Pantothenate |
|---|---|---|
| Strain | (660 nm) | g/l |
| MG442/pFV31ilvGM | 2.7 | 1.35 |
| MG442ΔpckA/ | 3.5 | 1.85 |
| pFV31ilvGM | | |

Modifications and Other Embodiments

Various modifications and variations of the described nucleic acids, plasmids, and cells as well as compositions and methods of using such products and the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the molecular biological, biochemical, chemical, chemical engineering, medical, or pharmacological arts or related fields are intended to be within the scope of the following claims.

Incorporation by Reference

Each document, patent application or patent publication cited by or referred to in this disclosure is incorporated by reference in its entirety. Any patent document to which this application claims priority is also incorporated by reference in its entirety. Specifically, priority documents DE 101 12 100.8, filed Mar. 14, 2001 and U.S. Provisional Application 60/304,774, filed Jul. 13, 2001 are hereby incorporated by reference.

What is claimed is:

1. Process for the preparation of D-pantothenic acid and/or alkaline earth metal salts thereof or feedstuffs additives comprising these by fermentation of microorganisms of the Enterobacteriaceae family, in particular those which already produce D-pantothenic acid, wherein
a) at least the nucleotide sequence(s) in the microorganisms which code(s) for the pckA gene is (are) attenuated, in particular eliminated,
b) the D-pantothenic acid and/or salts thereof is (are) concentrated in the medium or in the cells of the microorganisms, and
c) after conclusion of the fermentation, the desired products are isolated, the biomass and/or further constituents of the fermentation broth being left in the product or optionally being separated off completely or in part.

2. Process according to claim 1, wherein the fermentation is carried out in the presence of alkaline earth metal salts, these being added continuously or discontinuously in stoichiometric amounts in particular, and a product comprising alkaline earth metal salts of D-pantothenic acid or consisting of these being obtained.

3. Process according to claim 1, wherein the microorganisms of the Enterobacteriaceae family belong to the genus Escherichia.

4. Process according to claim 3, wherein the microorganisms originate from the genus Escherichia, in particular the species Escherichia coli.

5. Process according to claim 1, wherein in addition to attenuation of the pckA gene, one or more genes, chosen from the group, is (are) enhanced, in particular over-expressed:
5.1 the ilvGM operon which codes for acetohydroxy-acid synthase II,
5.2 the panB gene which codes for ketopantoate hydroxymethyl transferase,
5.3 the panE gene which codes for ketopantoate reductase,
5.4 the panD gene which codes for aspartate decarboxylase,
5.5 the panC gene which codes for pantothenate synthetase,
5.6 the serC gene which codes for phosphoserine transaminase,
5.7 the gcvT, gcvH and gcvP genes which code for the glycine cleavage system, individually or together,
5.8 the glyA gene which codes for serine hydroxymethyl transferase.

6. Process according to claim 1, wherein bacteria are employed in which the metabolic pathways which reduce the formation of D-pantothenic acid are at least partly eliminated, in particular the
6.1 avtA gene which codes for transaminase C and
6.2 the poxB gene which codes for pyruvate oxidase.

7. Process according to claim 1, wherein the expression of the polynucleotide (s) which code(s) for the pckA gene is attenuated, in particular eliminated.

8. Process for the preparation of feedstuffs additives comprising D-pantothenic acid and/or salts thereof, according to claim 1, wherein
a) optionally all or some of the biomass and/or a portion of the constituents are separated off from a D-pantothenic acid-containing fermentation broth obtained by fermentation,
b) the mixture obtained in this way is optionally concentrated, and
c) the feedstuffs additive comprising the pantothenic acid and/or the pantothenate is converted into a free-flowing form by suitable measures, and
d) a free-flowing animal feedstuffs additive with a particle size distribution of 20 to 2000 μm is obtained by suitable measures.

9. Process for the preparation of animal feedstuffs additives according to claim 8 with a content of D-pantothenic acid and/or salts thereof, chosen from the group consisting of the magnesium or calcium salt, in the range from about 20 to 80 wt. % (dry mass) from fermentation broths, comprising the steps of
a) optionally removal of water from the fermentation broth (concentration).
b) removal of an amount of ≧0 to 100% of the biomass formed during the fermentation,
c) optionally addition of one or more of the compounds mentioned to the fermentation broths obtained according to a) and b), the amount of compounds added being such that the total concentration thereof in the animal feedstuffs additive is in the range from about 20 to 80 wt. %, and
d) obtaining of the animal feedstuffs additive in the desired powder or, preferably, granule form.

10. Process according to claim 8, wherein an animal feedstuffs additive with the desired particle size is obtained from the fermentation broth, optionally after addition of D-pantothenic acid and/or salts thereof and optionally after addition of organic and inorganic auxiliary substances, by
a) drying and compacting, or
b) spray drying, or
c) spray drying and granulation, or
d) spray drying and build-up granulation.

11. Microorganisms of the Enterobacteriaceae family which produce pantothenic acid and in which the pckA gene is present in attenuated form.

12. Microorganisms of the Enterobacteriaceae family which produce pantothenic acid and in which the pckA gene is present in eliminated form.

13. Microorganisms of the Enterobacteriaceae family which produce pantothenic acid, according to claim 12, wherein the pckA gene contains a deletion according to SEQ ID No. 8.
